(19) United States
(12) Patent Application Publication
(10) Pub. No.: US 2018/0161138 A1
(43) Pub. Date: Jun. 14, 2018

(54) VAGINAL BAND SYSTEM

(71) Applicant: Ernesto Andrade, Bogota (CO)

(72) Inventor: Ernesto Andrade, Bogota (CO)

(21) Appl. No.: 15/378,014

(22) Filed: Dec. 13, 2016

(51) Int. Cl. A61F 2/00

(52) U.S. Cl. CPC A61F 2/0036; A61F 2220/0008

(57) ABSTRACT

A device and system to improve sexual intercourse by adjusting the diameter of the vaginal channel. In one embodiment, a vaginal strap comprises an implantable elongated semi-flexible biocompatible strap having a first and a second side. The strap further comprises a ribbed section, a middle section and a fastener section. The ribbed section comprises a plurality of parallel ribs and said fastener section comprises a fastener. The ribbed section engages said fastener, resulting in a circular loop that reduces vaginal diameter when implanted in a patient.

### BACKGROUND OF THE INVENTION

### Field of the Invention

The present invention relates to surgical implants, and more specifically to a resilient implant to be surgically implanted within the walls of the vagina to improve or restore sexual function.

### Background

Adequate friction during sexual intercourse is necessary in order to generate adequate sensorial stimulus in the vagina as well as the penis. It is easy to understand that, in cases where the penis is larger than the vagina, excess friction and pain results in less pleasure during sexual intercourse. At the same time, it is understandable that if the vaginal canal is too large in relation to the size of the penis, there would be insufficient friction to result in provide the expected pleasure from sexual intercourse. An increase in the diameter of the vaginal canal with respect to the diameter of the penis can be a source of stress for couples.

Loosening of the vaginal channel is normal in adult women due to childbirth, age, weight gain, lack of exercise, and muscular disorders. However, in some instances the vaginal channel becomes too loose inhibiting sexual intercourse which requires friction with the penis. Loosening the musculature of the pelvic floor and ligaments supporting the bladder are subject to significant stress during childbirth. There are well-known methods to assist in addressing this problem. For example, in U.S. Pat. No. 6,645,137 a vaginal insert with a flexible body is coiled and placed inside a patient's vagina. The device uncoils applying pressure against the vaginal walls. Most methods and devices directed to address the change in the size of the vaginal canal with respect to penis size relate to devices for external and removable use.

### SUMMARY OF THE INVENTION

It is one object of the present invention to provide a device and system to improve sexual intercourse by adjusting the diameter of the vaginal channel. In one embodiment, a vaginal strap comprises an implantable elongated semi-flexible biocompatible strap 100 having a first 104 and a second side 106. The strap 100 further comprises a ribbed section 108, a middle section 110 and a fastener section 114. The ribbed section 108 comprises a plurality of parallel ribs 116 and said fastener section comprises a fastener 120. The ribbed section 108 engages said fastener 120, resulting in a circular loop that reduces vaginal diameter when implanted in a patient.

In a further aspect of the invention, a vaginal band system comprises two or more vaginal straps, preferably three straps, wherein each vaginal strap comprises an implantable elongated semi-flexible biocompatible strap 100 having a first 104 and a second side 106. The strap 100 further comprises a ribbed section 108, a middle section 110 and a fastener section 114. The ribbed section 108 comprises a plurality of parallel ribs 116 and said fastener section comprises a fastener 120. The ribbed section 108 engages said fastener 120, resulting in a circular loop that reduces vaginal diameter when implanted in a patient.

An additional embodiment of the present invention provides a method for implanting a vaginal band system. In a first step, an anesthetic solution is administered to a patient. In a second step, a suture guide is inserted into a patient's vagina. In a third step, a swab loaded with tint is inserted into the suture guide, wherein the tint in the swab marks a location for insertion of a vaginal strap. In a subsequent step, the swab is removed. In another step, the vaginal strap in is implanted in the patient's vagina utilizing marks generated by the tint. In a final step the vaginal strap is secured in place.

The proposed device is a permanent, implantable, semi-elastic insert that provides for maintaining a constant diameter of the vaginal canal and improves friction between the vaginal walls and the penis during sexual intercourse resulting in increased pleasure for both partners.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 is a perspective view of one embodiment of the present invention.

FIG. 2 is a top view of one embodiment of the present invention.

FIG. 3 is a perspective view of a fastener in accordance with one embodiment of the present invention.

FIG. 4 is a perspective view of the engagement member fastened to the fastener in accordance with one embodiment of the present invention.

FIG. 5A is a diagram showing a vaginal canal of a patient.

FIG. 5B is a diagram showing a suture guide used in preparing the vaginal canal for implantation of a vaginal strap in accordance with one embodiment of the present invention.

FIG. 5C is a diagram showing the suture guide inserted in the vaginal canal in accordance with one embodiment of the present invention.

FIG. 5D is a diagram showing a swab and the suture guide inserted in the vaginal canal in accordance with one embodiment of the present invention.

FIG. 5E is a diagram showing a swab inserted in the suture guide, which is inserted in the vaginal canal in accordance with one embodiment of the present invention.

FIG. 5F is a diagram showing a swab removed from the suture guide, which is inserted in the vaginal canal in accordance with one embodiment of the present invention.

FIG. 5G is a diagram showing the suture guide removed from the vaginal canal leaving in place markers in accordance with one embodiment of the present invention.

FIG. 6A through 6I is a diagram showing the various steps in the implantation of the vaginal strap in accordance with one embodiment of the present invention. FIG. 6A shows the position of marks around the vaginal canal. FIGS. 6B, 6C, 6D, 6E, 6F, 6G, 6H, 6I show how a vaginal strap is implanted into the vaginal canal of a patient.

FIG. 7 is a perspective view of one embodiment of the present invention.

### DETAILED DESCRIPTION

The invention summarized above and defined by the enumerated claims may be better understood by referring to the following description, which should be read in conjunction with the accompanying drawings in which like reference numbers are used for like parts. This description of an embodiment, set out below to enable one to build and use an implementation of the invention, is not intended to limit the invention, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

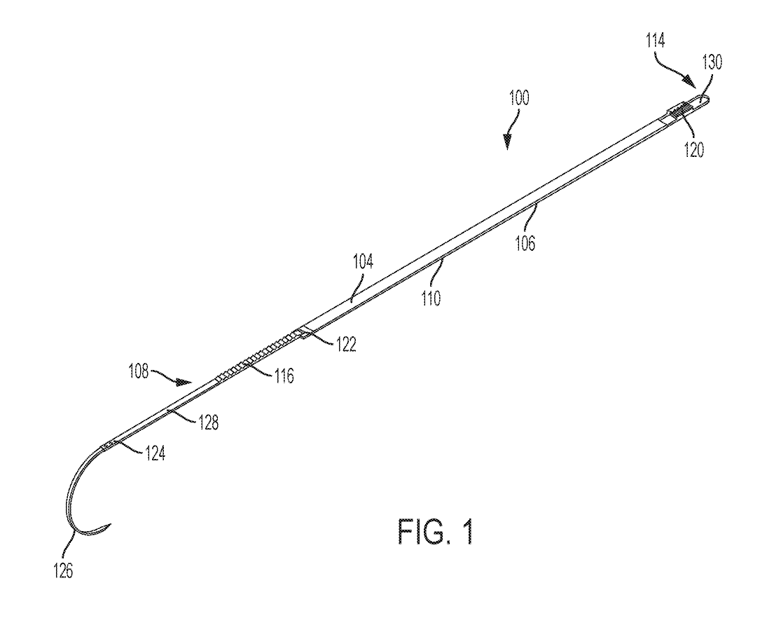

One embodiment of the present invention as shown in FIG. 1 is a vaginal strap that comprises an implantable elongated semi-flexible biocompatible strap 100 having a first 104 and a second side 106. The strap 100 is made from medical grade silicone or other similar biological compatible material. The strap 100 further comprises a ribbed section 108, a middle section 110 and a fastener section 114. The ribbed section 108 comprises a plurality of parallel ribs 116 and the fastener section comprises a fastener 120. In a preferred embodiment, the ribbed section 108 engages said fastener 120 resulting in a circular loop that reduces vaginal diameter when implanted in a patient.

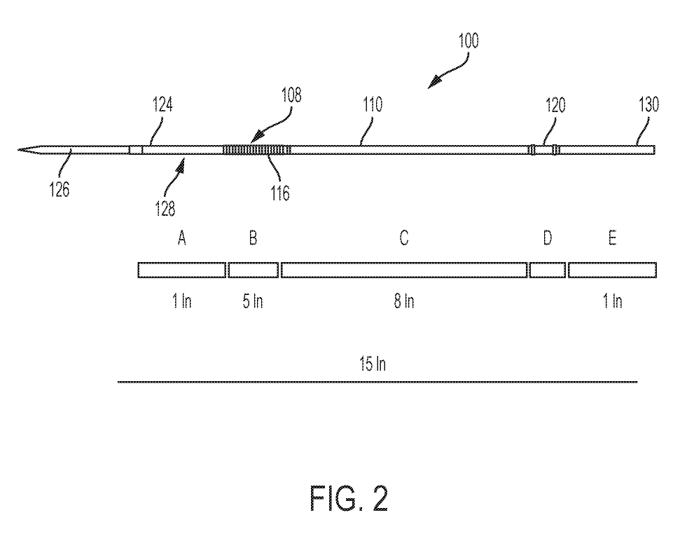

In another preferred embodiment, the ribbed section 108 comprises a middle section end 122 and a needle section end 124 and it further comprises a needle 126 attached to said needle section end 124. The needle 126 is made of malleable stainless steel. The ribbed section 108 further comprises an engagement member 128 without parallel ribs and the fastener section 114 has an elongate extension 130. As shown in FIG. 2, the vaginal strap 100 in one exemplary embodiment is 15 inches long. Where the fastener section is 1 inch in length, the middle section 110 is 8 inches in length. The ribbed section 108 is 5 inches in length and the engagement member 128 is 1 inch in length.

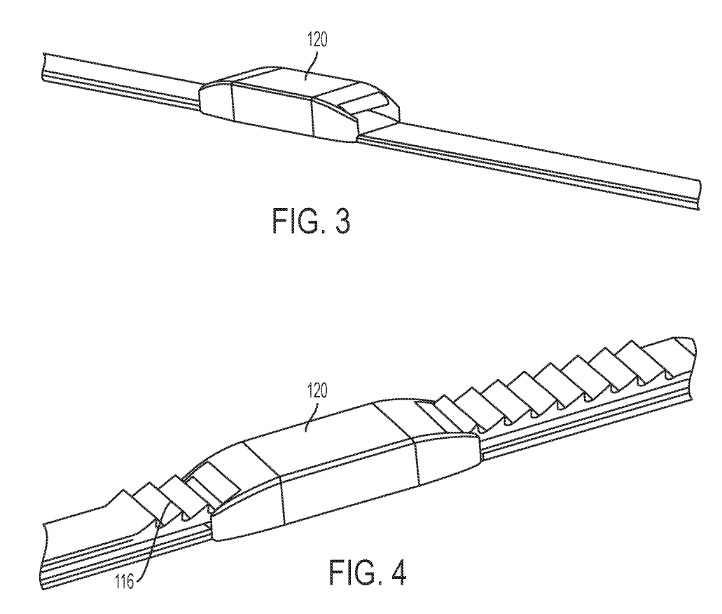

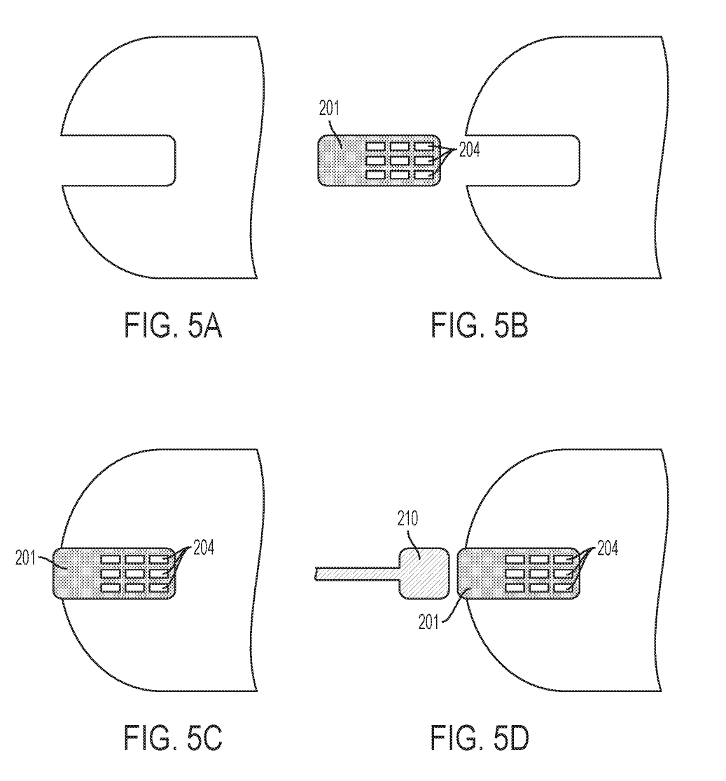

The strap 100 can be used in a vaginal band system. The system includes two or more vaginal straps. Each vaginal strap comprises an implantable elongated semi-flexible biocompatible strap 100 having a first 104 and a second side 106. The strap 100 further comprises a ribbed section 108, a middle section 110 and a fastener section 114. The ribbed section 108 comprises a plurality of parallel ribs 116, and said fastener section comprises a fastener 120 (shown in more detail in FIG. 3), wherein said ribbed section 108 engages said fastener 120 as shown in FIG. 4, resulting in a circular loop that reduces vaginal diameter when implanted in a patient. In other embodiments, the system has at least three vaginal straps.

In a further embodiment, the system includes an installation kit. The installation kit has a suture guide 201 with a plurality of perforations 204 concentrically aligned. The suture guide 201 is a plastic cylinder made from biological compatible materials, preferably hypoallergenic. In a further embodiment, the system also includes a swab 210 and marking tint.

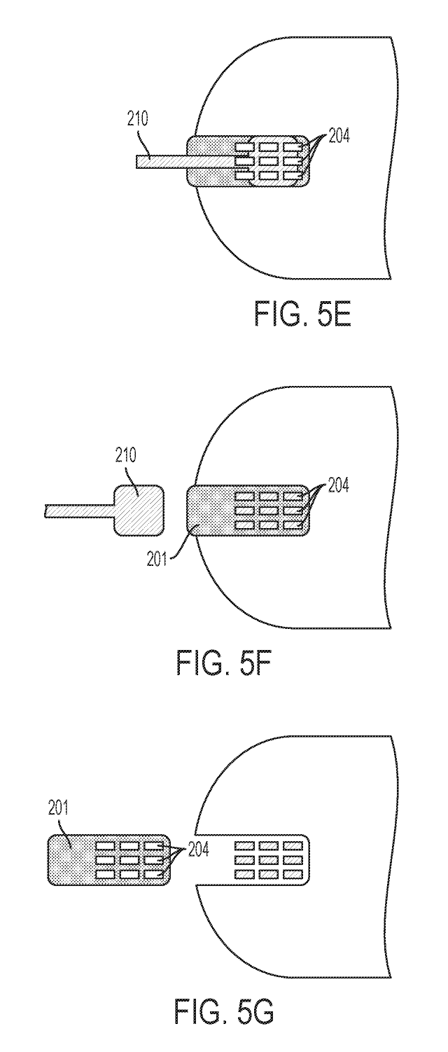

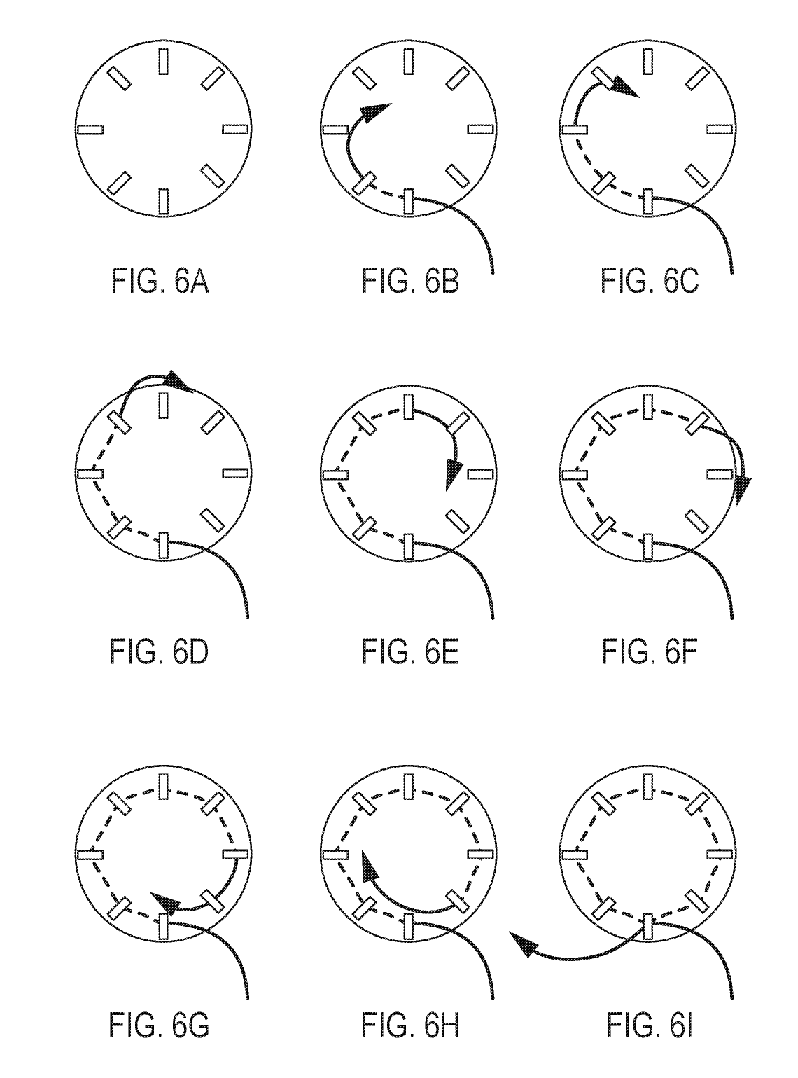

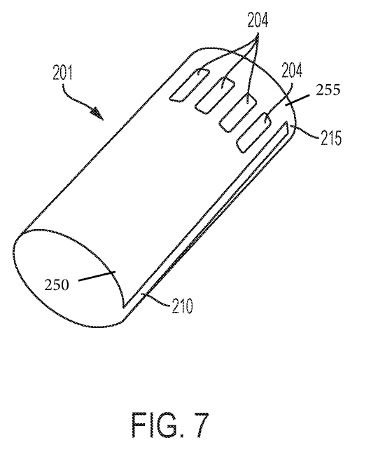

The vaginal band system is implanted in a patient, using the method shown in FIGS. 5A to 5G. First, an anesthetic solution is provided to the patient. It is understood that the anesthetic solution can be a local anesthetic placed in the patient's vagina. The anesthetic solution may also be a solution used in an appropriate general anesthesia protocol. In a second step, the suture guide 201 (which is shown in more detail in FIG. 7) is inserted into the patient's vagina as shown in FIG. 5C. During this step, the vaginal channel is opened utilizing appropriate surgical instruments. For example, vaginal forceps can be utilized to open the vaginal channel allowing the surgeon to complete the procedure. In another step, a swab 210 loaded with tint is inserted into the suture guide 201, wherein the tint in the swab marks a location for insertion of a vaginal strap as shown in FIGS. 5D and 5E. In a further step, the swab 210 is removed as shown in FIG. 5F and then, or simultaneously, the suture guide 201 as shown in FIG. 5G. The vaginal strap 100 is then implanted in the patient's vagina utilizing marks generated by the tint. The implant sequence is shown on FIGS. 6A through 6I. Finally, the vaginal strap is secured in place by removing the needle, inserting the engagement member 128 into the fastener 120.

In one preferred embodiment, as show on FIG. 7, the suture guide 201 includes a channel 210. The channel 210 extends from first end 250 to a second end 255. In such embodiment, the diameter of the suture guide can increase or decrease because the edges of the channel 210 are free to overlap. In a further embodiment, the channel 210 extends from the first end 250 towards the second end 255, but it stops at bridge 215. The bridge 215 prevents the channel edges from overlapping. The channel 210 is used during implantation to maintain the suture thread in the correct position. The suture guide 201 can be utilized during implantation. Initially, the suture guide 201 is positioned so that the first end 250 is closest to the vaginal opening, while the second end 255 is closest to the uterine wall. The channel 210 is positioned at the lowest position, which is closest to the anal canal. The openings 204 are marked as discussed above. The guide 201 is then utilized to assist the surgeon in the process of implanting the vaginal straps 100. The strap 100 is inserted through a first opening 204, the guide is then turned to the next opening with the suture thread along the channel 210. The strap 100 is then inserted in the next opening 204, and the guide is turned again to the next opening. The process continues until the guide reaches the initial position and the surgeon can then close the strap 100.

In another exemplary embodiment of the method, at least two vaginal straps are implanted in the patient, preferably three straps. The vaginal strap 100 is then adjusted to have a specific diameter. The vaginal strap comprises a needle for implantation, which needle is removed from the vaginal strap once a circular loop is formed. In one preferred embodiment of the method, the ribbed section and fastener section are joined utilizing two needle carriers. In a final step, a suture is applied in order to close any lacerations in vaginal region. Once implanted, the vaginal strap has a tolerance of elasticity that allows it to increase its diameter by up to 40%.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

What is claimed is:

1. A vaginal strap, comprising:
an implantable elongated semi-flexible biocompatible strap having a first and a second side,
said strap further comprising a ribbed section, a middle section and a fastener section,
said ribbed section comprises a plurality of parallel ribs, and said fastener section comprises a fastener, wherein said ribbed section engages said fastener, resulting in a circular loop that reduces vaginal diameter when implanted in a patient.

2. The vaginal strap of claim 1, wherein the ribbed section comprises a middle section end and a needle section end and it further comprises a needle attached to said needle section end.

3. The vaginal strap of claim 2, wherein said needle is made of malleable stainless steel.

4. The vaginal strap of claim 1, wherein the ribbed section further comprises an engagement member without parallel ribs.

5. The vaginal strap of claim 1, wherein said fastener section has an elongate extension.

6. The vaginal strap of claim 1, wherein the strap is made from medical grade silicone.

7. A vaginal band system, comprising:
two or more vaginal straps, wherein each vaginal strap comprises an implantable elongated semi-flexible biocompatible strap having a first and a second side,
said strap further comprising a ribbed section, a middle section and a fastener section,
said ribbed section comprises a plurality of parallel ribs, and said fastener section comprises a fastener, wherein said ribbed section engages said fastener, resulting in a circular loop that reduces vaginal diameter when implanted in a patient.

8. The system of claim 7, comprising at least three vaginal straps.

9. The system of claim 7, further comprising an installation kit comprising:
a suture guide.

10. The system of claim 9, wherein the suture guide, comprises a plurality of perforations concentrically aligned.

11. The system of claim 9, wherein the suture guide is a plastic cylinder.

12. The system of claim 7, further comprising a swab and marking tint.

13. A method for implanting a vaginal band system comprising:
administering an anesthetic solution;
inserting a suture guide into a vagina;
inserting a swab loaded with tint into the suture guide, wherein the tint in the swab marks a location for insertion of a vaginal strap, and removing said swab and suture guide;
inserting the vaginal strap in the patient's vagina utilizing marks generated by the tint,
securing the vaginal strap in place.

14. The method of claim 13, wherein at least three vaginal straps are implanted in the patient.

15. The method of claim 13 further comprising adjusting the vaginal strap to have a specific diameter.

16. The method of claim 13, wherein the vaginal strap comprises a needle for implantation of the vaginal strap, which needle is removed from the vaginal strap once a circular loop is formed.

17. The method of claim 16, wherein a ribbed section of the vaginal strap is fastened to a fastener section of the vaginal strap.

18. The method of claim 17, wherein said ribbed section and fastener section are joined utilizing two needle carriers.

19. The method of claim 17, further comprising the step of applying a suture after the vaginal strap is implanted.

20. The method of claim 17, wherein the vaginal strap has a tolerance of elasticity that allows it to increase its diameter by up to 40%.
